(19) United States
(12) Patent Application Publication
(10) Pub. No.: US 2019/0274902 A1
(43) Pub. Date: Sep. 12, 2019

(54) Integrated temperature sensitive wound dressing device

(71) Applicants: Anthony V Beran, Yorba Linda, CA (US); Kerry J Tomic-Edgar, Yorba Linda, CA (US)

(72) Inventors: Anthony V Beran, Yorba Linda, CA (US); Kerry J Tomic-Edgar, Yorba Linda, CA (US)

(21) Appl. No.: 15/932,501

(22) Filed: Mar. 7, 2018

(51) Int. Cl. A61F 13/84; A61F 13/00; G01K 13/00; A61B 5/01; G01K 11/12

(52) U.S. Cl. CPC A61F 13/84; A61F 13/00059; A61M 2025/0246; A61B 5/01; G01K 11/12; G01K 13/002

(57) ABSTRACT

The present invention pertains to an integrated wound dressing device for treatment of an insertion site of percutaneous and drug delivery devices. The integrated wound dressing device preferably comprises a transparent film layer having a bottom side, a top side and a perimeter. The bottom side is coated with an adhesive impregnated with an antimicrobial agent. The transparent film has a radius cutout between a central opening and the perimeter. A liquid crystal temperature sensitive film with an adhesive placed on the top side of the transparent film layer is preferably near and around the central opening. The dressing also has a layer of double folded release paper below the bottom side of the transparent film layer. The preferred TLCs used are cholesteryl esters with a preferable temperature detection range of 35-40° C. The preferred antimicrobial agent is chlorohexidine gluconate (CHG).

### TECHNICAL FIELD

The present invention pertains to an integrated wound dressing device for treatment of an insertion site of percutaneous and drug delivery devices.

### BACKGROUND ART

Intravascular (IV) catheters are widely used in clinical situations. All patients with IV catheters risk developing complications related to catheter placement. Among the many complications reported, infection is the most problematic. When skin is compromised, such as during catheter insertion, it provides a path for bacteria to migrate along the catheter wall and cause an infection. This bacterial migration is further facilitated by catheter movement and micropistoning. The estimates of the occurrences of Central Line Blood Stream Infections (CLABSIs) in the United States is approximately 250,000 cases per year. These infections are associated with a high mortality rate (12-25%) and extended hospital stays of an additional 9-12 days. This can result in an increase of the cost of treatment by $34,000-$56,000 per patient.

Recently, a product that incorporates antimicrobial properties and is placed around the catheter insertion site, has been introduced in the clinical practice. The product is design to decrease bacterial flora on the skin surface around the catheter insertion site. The product comes in a form of circular patch made of synthetic and biopolymer composite foam/sponge impregnated with Chlorohexidine Gluconate (CHG). The product is referred to by its trademark, BIOPATCH™. However, BIOPATCH™ is non-transparent and it does not have an adhesive on its lower/patient side. It is secured around the insertion site by secondary transparent film with adhesive. A transparent film dressing that permits a visual observation of catheter insertion site is disclosed in U.S. Pat. No. 5,372,589. The product utilizing this patent is sold under the trademark SorbaView SHIELD™, but it is for a transparent dressing alone. U.S. Pat. No. 5,833,665 discloses a non-transparent foam pad with an adhesive layer on the bottom/patient side. U.S. Pat. No. 8,969,649 discloses a transparent film with acrylic adhesive impregnated with CHG

The use of these commercially available products has decreased the potential for infection, but it could be further decreased. One way to achieve a further decrease is to detect a local infection at its very beginning stage before it spreads systemically. The first sign of local infection is often local skin redness accompanied with elevated skin temperature around the insertion site. Thus, a device that aids in early detection of infections at catheter insertion sites is desired.

### SUMMARY OF THE INVENTION

The present invention pertains to an integrated wound dressing device for treatment of an insertion site of percutaneous and drug delivery devices. The integrated wound dressing device preferably comprises a transparent film layer having a bottom side, a top side and a perimeter. The bottom side is coated with an adhesive impregnated with an antimicrobial agent. The transparent film has a radius cutout between a central opening and the perimeter. A liquid crystal temperature sensitive film with an adhesive placed on the top side of the transparent film layer is preferably near and around the central opening. The dressing also has a layer of double folded release paper below the bottom side of the transparent film layer. The preferred TLCs used are cholesteryl esters with a preferable temperature detection range of 35-40° C. The preferred antimicrobial agent is chlorohexidine gluconate (CHG).

### BRIEF DESCRIPTION OF THE DRAWINGS

The objects and features of the present invention, which are believed to be novel, are set forth with particularity in the appended claims. The present invention, both as to its organization and manner of operation, together with further objects and advantages, may best be understood by reference to the following description, taken in connection with the accompanying drawings.

FIG. 1A is a top view of a preferred embodiment of the invention;

FIG. 1B is a side view of the embodiment in FIG. 1A;

FIG. 2A is a top view of another embodiment of the invention;

FIG. 2B is a side view of the embodiment in FIG. 2A;

FIG. 3A is a top view of another embodiment of the invention;

FIG. 3B is a side view of the embodiment in FIG. 3A;

FIG. 4A is a top view of another embodiment of the invention;

FIG. 4B is a side view of the embodiment in FIG. 4A;

FIG. 5A is a top view of another embodiment of the invention;

FIG. 5B is a side view of the embodiment in FIG. 5A;

FIG. 6A is a top view of another embodiment of the invention;

FIG. 6B is a side view of the embodiment in FIG. 6A;

FIG. 7A is a top view of another embodiment of the invention;

FIG. 7B is a side view of the embodiment in FIG. 7A;

FIG. 8A is a top view of another embodiment of the invention;

FIG. 8B is a side view of the embodiment in FIG. 8A;

FIG. 9A is a top view of another embodiment of the invention; and,

FIG. 9B is a side view of the embodiment in FIG. 9A.

### DETAILED DESCRIPTION OF THE PREFERRED EMBODIMENTS

Various embodiments are now described with reference to the drawings, wherein like reference numerals are used to refer to like elements throughout. In the following description, for purposes of explanation, numerous specific details are set forth in order to provide a thorough understanding of one or more embodiments. It may be evident, however, that such embodiment(s) may be practiced without these specific details.

In the following paragraphs, the present invention will be described in detail by way of example with reference to the attached drawings. Throughout this description, the preferred embodiment and examples shown should be considered as exemplars, rather than as limitations on the present invention. As used herein, the “present invention” refers to any one of the embodiments of the invention described herein, and any equivalents. Furthermore, reference to various feature(s) of the “present invention” throughout this document does not mean that all claimed embodiments or methods must include the referenced feature(s). The following description is provided to enable any person skilled in the art to make and use the invention and sets forth the best modes contemplated by the inventors of carrying out their invention. Various modifications, however, will remain readily apparent to those skilled in the art, since the general principles of the present invention have been defined herein specifically to provide an improved integrated wound dressing device for treatment of an insertion site of percutaneous and drug delivery devices.

The invention preferably incorporates Temperature Sensitive Liquid Crystal (TLC) carrier with antimicrobial carrier, for early detection of local infection, by displaying changes in skin temperature around catheter insertion site. This integrated device preferably provides: a delivery of antibacterial agent around the catheter insertion site, a removal of exudate from around the insertion site, and a detection of changes in skin temperature around the insertion site.

Delivery of antimicrobial agents is preferably obtained by incorporating substances like CHG with sponge like materials, copolymers films or castings and adhesives. Some examples of other antimicrobial agents that can be used are: Chlorohexidine acetate, silver iodine, silver bromide, nano-particulate metallic silver, benzo conium chloride, and triclosone.

Removal of exudate is preferably done by selecting a transparent film or casting that has the most adequate ratio of static fluid absorption and gas/vapor transmission.

Change in temperature is preferably observed by placing TLC film in different areas over the antimicrobial transparent film, or by mixing non-toxic mixtures of TLC with CHG within co-polymers or adhesives. The TLC, in order to function properly, is preferably protected from the environment. This protection can be achieved by:
A. TLC dispersion: TLC is dispersed as a discrete aggregate into the polyurethane materials such as polyurethane elastomers and polyurethane polymers. Films, sheets, castings or other three-dimensional forms of compounded mixtures, following proper polymerization, can be used as a temperature indicator. By properly selecting polyurethane for sponge formulation, the TLC can be incorporated into sponge structure. Or,
B. TLC microencapsulation: TLC is treated by microencapsulation process to produce microencapsulated discrete globs of liquid crystal, each coated separately, which then can be incorporated into a carrier of selective functions.

The preferred urethane compounds for this invention are polyols, polyamines, polyamides or other suitable compounds. The preferred TLCs used are cholesteryl esters such as chloride, iodide, bromide, cinnamate, oleyl carbonate, acetate, nonanoate, hexanoate, linolineate, oleate, laurate, caproate, myristrate, hydrogen phthalate, benzoate, and others disclosed in U.S. Pat. No. 3,872,050. Different temperature ranges can be detected using different mixtures or rations of several TLC compounds in a single formulation or using a multilayer dispersion of several specific TLC. The temperature range is preferably 35-40 degrees Celsius (° C.).

To obtain better visibility of changes in colors, as a result in changes in temperature, the following can be performed: 1.) Polymerized temperature sensitive compound can be deposited on a dark background film; 2.) A carbonaceous black powder can be mixed during the TLC dispersion process; or, 3.) carbonaceous powder can be mixed during the microencapsulation process.

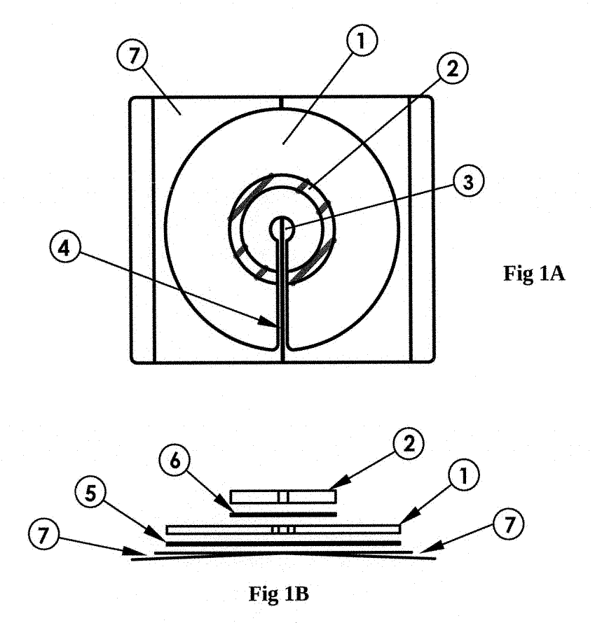

Referring now to FIG. 1A, a top view of the invention is shown. A gas and water vapor permeable transparent film 1 is shown. The bottom side of the film 1 is preferably coated with an acrylic adhesive impregnated with/permeated by an antimicrobial agent. A common example of such a film with antimicrobial adhesive is Bene Hold™ CHG transparent dressing. The bottom side is also referred to as the “patient side” because it contacts the patient's skin. The top side of the transparent film 1 is preferably smooth and faces away from the patient. Preferably, the transparent film 1 has a circular shape with an opening 3 placed in the center of the circle. As shown, a liquid crystal temperature sensitive film 2 with an adhesive, such as Edmund Optics™ liquid crystal sheet, having a 35-40° C. range, is placed centrally around the opening 3 on the top side of the transparent film 1. A radius cutout 4 preferably provides a passage between the center cutout 3 and the perimeter of the transparent film 1. The transparent film 1 is preferably placed over two pieces of double folded released paper 7 with two internal edges of a release paper folds, being aligned with the radius cutout 4, for easy placement of the invention around a catheter insertion site (not shown). In application, the radius cutout 4 is slid along the side of the catheter (not shown), until the catheter extends through the center cutout 3. The wings of double folded release paper 7 are then pulled off to expose the adhesive on the bottom side so that the invention can be secured to the skin of the patient.

[12] Referring now to FIG. 1B, a side view of the embodiment shown in FIG. 1A is shown. As discussed above, the transparent film 1 is preferably covered with an antimicrobial adhesive 5 and placed over the two parts of a double folded release paper 7. The liquid crystal temperature sensitive film 2 with adhesive 6 on its bottom surface is placed on the top surface of the transparent film 1. The liquid crystal film 2, preferably in the form of a ring, is placed around center cut out 3. In this configuration, the changes in temperature are observed 360 degrees around a catheter insertion site and in proximity to the insertion site. Preferably, there is space between the inner circumference of the liquid crystal film 2 and the central opening 3 and, thus, the outer circumference of the catheter (not shown) for visual observation of the patient's insertion site.

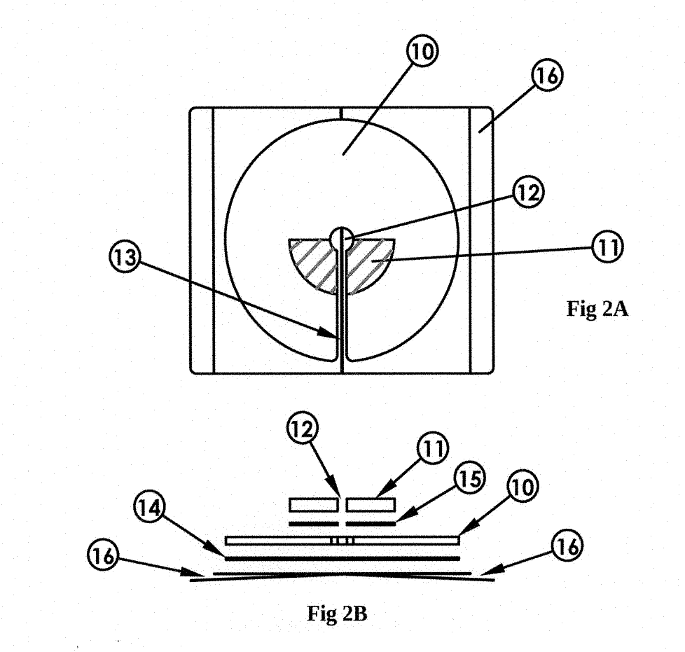

Referring now to FIG. 2A, a top view of another embodiment of the present invention is shown. A gas and water vapor permeable transparent film 10 is shown. The bottom side of the film 10 is preferably coated with an acrylic adhesive impregnated with/permeated by an antimicrobial agent. The top side of the transparent film 10 is preferably smooth and faces away from the patient. Preferably, the transparent film 10 has a circular shape with an opening 12 placed in the center of the circle. As shown, a liquid crystal temperature sensitive film 11 with an adhesive is placed centrally half way around the opening 12 on the top side of the transparent film 10. A radius cutout 13 preferably provides a passage between the center cutout 12 and the perimeter of the transparent film 10. The transparent film 10 is preferably placed over two pieces of double folded released paper 16 with two internal edges of a release paper folds, being aligned with the radius cutout 13, for easy placement of the invention around a catheter insertion site (not shown).

Referring now to FIG. 2B, a side view of the embodiment shown in FIG. 2A is shown. As discussed above, the transparent film 10 is preferably covered with an antimicrobial adhesive 14 and placed over the two parts of a double folded release paper 16. The liquid crystal temperature sensitive film 11 with adhesive 15 on its bottom surface is placed on the top surface of the transparent film 10. The liquid crystal film 11, preferably in the form of a half ring, is placed around center cut out 12. In this configuration, the changes in temperature are observed 180 degrees around a catheter insertion site and in proximity to the insertion site while the other 180 degrees provides for visible observation of the insertion site.

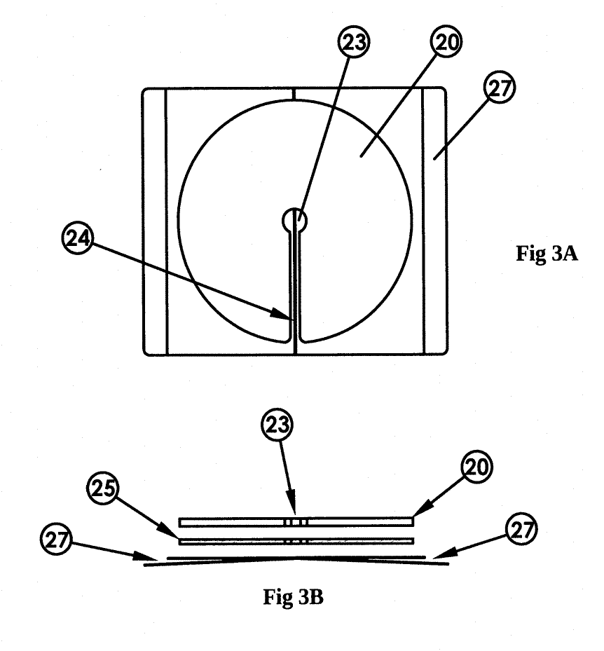

Referring now to FIG. 3A, a top view of another embodiment of the present invention is shown. In this embodiment, a transparent film 20 has its bottom surface covered with an acrylic adhesive impregnated or mixed with an antimicrobial agent and a non-toxic mixture of liquid crystal esters, providing for observation of temperature changes by observing the changes in color over the entire transparent film area. Preferably, the transparent film 20 has a circular shape with an opening 23 placed in the center of the circle. A radius cutout 24 preferably provides a passage between the center cutout 23 and the perimeter of the transparent film 20. The transparent film 20 is preferably placed over two pieces of double folded released paper 27 with two internal edges of a release paper folds, being aligned with the radius cutout 24, for easy placement of the invention around a catheter insertion site (not shown). A visual observation of the insertion site is limited with this embodiment because microencapsulatcd globes of TLC, which are dispersed through the acrylic adhesive, are preferably coated with the carrier mixed with black powder.

Referring now to FIG. 3B, a side view of the embodiment shown in FIG. 3A is shown. As discussed above, the transparent film 20 is preferably covered with an antimicrobial adhesive mixed with microencapsulated globs of TLC 25 and placed over the two parts of a double folded release paper 27.

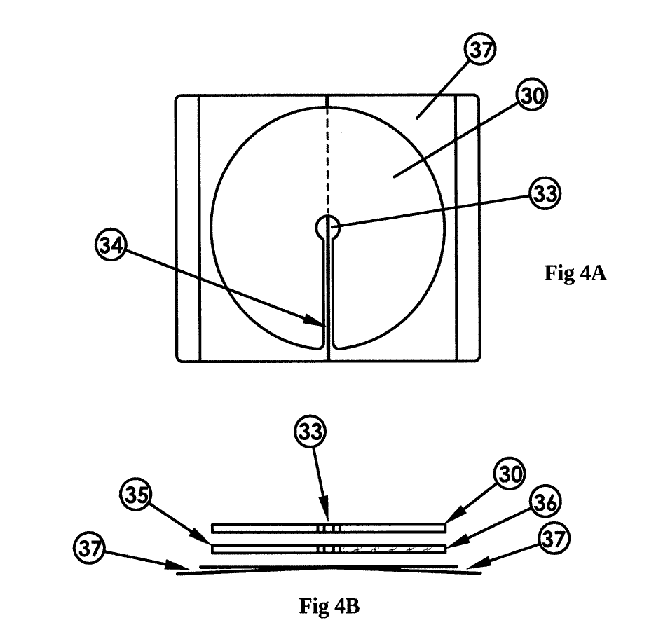

Referring now to FIG. 4A, a top view of another embodiment of the present invention is shown. A gas and water vapor transparent film 30 is shown. The bottom side of the film 30 is preferably coated with two types of acrylic adhesive, the adhesives are preferably separated along the radius cutout 33. Preferably, the transparent film 30 has a circular shape with an opening 33 placed in the center of the circle. A radius cutout 34 preferably provides a passage between the center cutout 33 and the perimeter of the transparent film 30. The transparent film 30 is preferably placed over two pieces of double folded release paper 37, with two internal edges of a release paper folds, being aligned with the radius cutout 34 for easy placement of the invention around a catheter insertion site (not shown).

Referring now to FIG. 4B, a side view of the embodiment shown in FIG. 4A is shown. As discussed above, the transparent film 30 is preferably covered with two types of adhesive. Side 35 shown in FIG. 4B is coated with an adhesive mixed with an antimicrobial agent. Side 36 shown in FIG. 4B is coated with an adhesive mixed with an antimicrobial agent and microencapsulated globs of TLC. This embodiment provides for a delivery of antimicrobial agent 360 degrees around the insertion site and 180 degrees temperature change visualization around the insertion site.

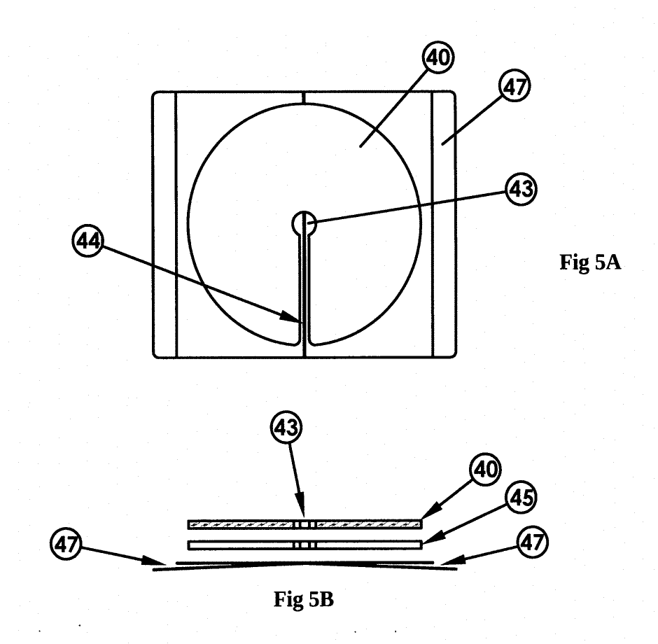

Referring now to FIG. 5A, a top view of another embodiment of the present invention is shown. In this embodiment, a transparent film or a copolymer cast 40 contains TLC as a dispersed aggregate. A change in color of the transparent film or cast 40 provides for the observation of temperature changes over the entire transparent film/cast areas. The transparent film or cast 40 has its bottom surface covered with an acrylic adhesive impregnated or mixed with an antimicrobial agent. Preferably, the transparent film/cast 40 has a circular shape with an opening 43, placed in the center of the circle. A radius cut out 44 preferably provides a passage between the center cutout 43 and the perimeter of the transparent film 40. The transparent film 40 is preferably placed over two pieces of double folded release paper 47, with two internal edges of a release paper folds, being aligned with the radius cutout 44, for easy placement of the invention around a catheter insertion site (not shown). Because the discrete aggregates of TLC, which are dispersed through the transparent film/cast 40, are also mixed with a black powder, a visual observation of the insertion site is limited.

Referring now to FIG. 5B, a side view of the embodiment shown in FIG. 5A, is shown. As discussed above, the transparent film/cast 40 is preferably covered on its patient side with an antimicrobial adhesive 45 and placed over the two parts of a double folded release paper 47.

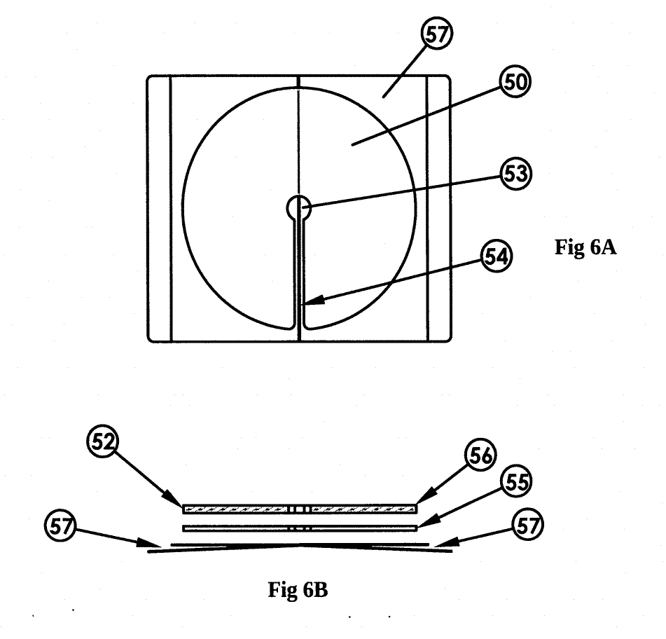

Referring now to FIG. 6A, a top view of another embodiment of the present invention is shown. A gas and water vapor transparent film/cast 50 is subdivided into two parts, which are separated along the radius cutout 54. The bottom side 55 of the film/cast 50 is coated with an acrylic adhesive impregnated/mixed with an antimicrobial agent. Preferably, the transparent film 50 has a circular shape with an opening 53 placed in the center of the circle. A radius cutout 54 preferably provides a passage between the center of the cutout 53 and the perimeter of the transparent fil/cast 50. The transparent film/cast 50 is preferably placed over the two pieces of double folded release paper 57 with 2 internal edges of a release paper folds being aligned with radius cutout 54 for easy placement of the invention around a catheter insertion site (not shown).

Referring now to FIG. 6B, a side view of the embodiment shown in FIG. 6A is shown. As discussed above, the transparent film/cast 50 is preferable subdivided into two sides. Side 52 is preferably transparent film/cast alone. Side 56 is preferably transparent film/cast with microencapsulated globes of TLC. This embodiment provide for a delivery of antimicrobial agent 360 degrees around the insertion site and provides for 180 degrees of temperature change visualization around the insertion site, and 180 degrees of visualization of the catheter insertion site.

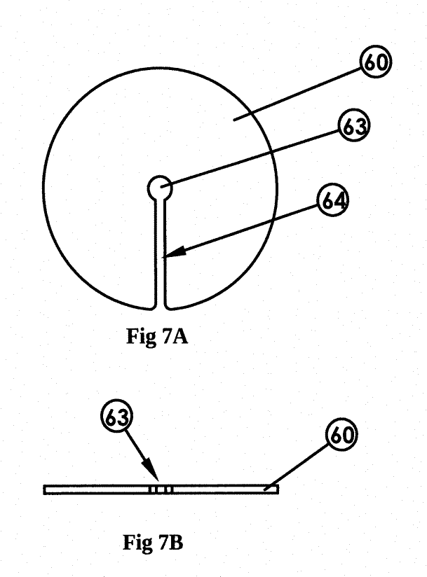

Referring now to FIG. 7A, a top view of another embodiment of the present invention is shown. In this embodiment a preferably non-transparent foam 60 without adhesive is formulated with a mixture of an antimicrobial agent and TLC or TLC alone, with antimicrobial agent being impregnated into the foam. There is a radius cutout 64 for sliding the foam around a catheter to place the catheter in the center cutout 63. The TLC allows the foam 60 to change color as an indicator of temperature changes. This color change is preferably observed over the entire surface of the circular foam 60. FIG. 7B, is a side view of this embodiment.

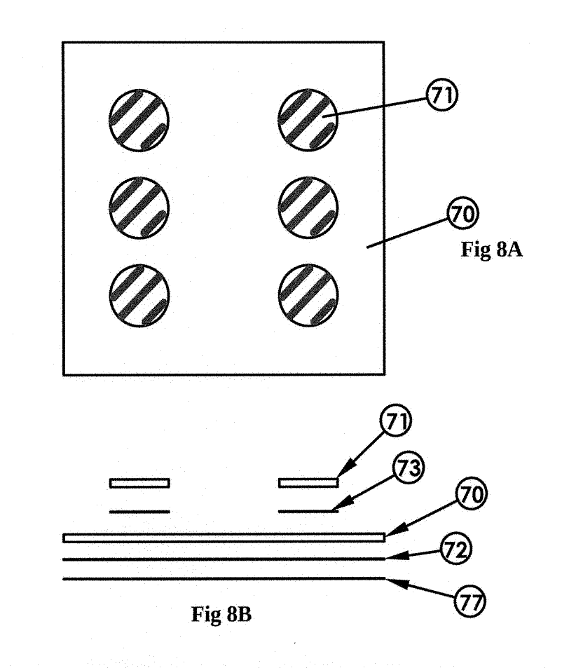

Referring now to FIGS. 8A and 8B, another embodiment of the present invention is shown. In this embodiment, a plurality of liquid crystal temperature sensitive films 71, preferably in the shape of a circle, are placed randomly or in a pattern over the surface of a gas and water vapor permeable transparent film 70. The transparent film 70 preferably has a smooth top surface and the bottom surface is coated with an acrylic adhesive impregnated with an antimicrobial agent 72. The TLC film 71 preferably has a smooth top surface and a bottom surface covered with an acrylic adhesive 73 to attach to the film 70. The transparent film 70 with its bottom surface adhesive 72 is attached to a release liner 77. A side view of the embodiment in FIG. 8A is shown in FIG. 8B.

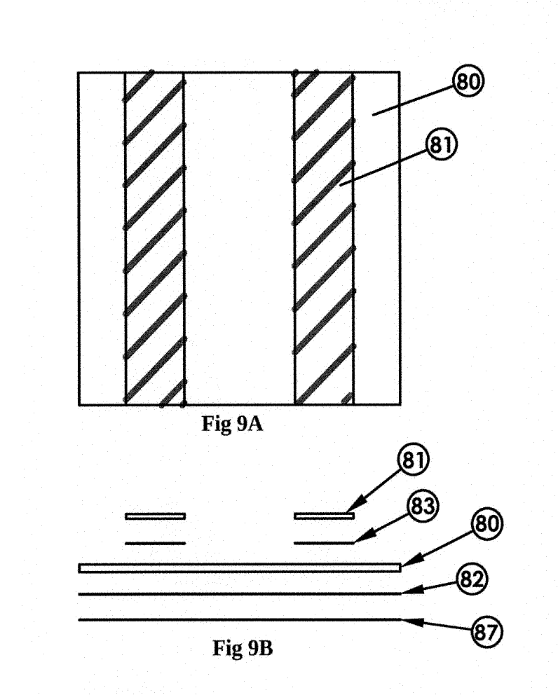

Referring now to FIG. 9A, a top view of another preferred embodiment of the invention is shown. In this embodiment, a liquid crystal temperature sensitive film TLC 81 is placed longitudinally or transversely on a gas and water vapor permeable transparent film 80. The transparent film 80 preferably has a smooth top surface and the bottom surface is coated with an acrylic adhesive impregnated with an antimicrobial agent 82. The liquid crystal temperature film 81 preferably has a smooth top surface and a bottom surface covered with an acrylic adhesive 83 to attach to the film 80. The adhesive 82 is covered with a release paper 87. A side view of the embodiment in FIG. 9A is shown in FIG. 9B.

Thus, an improved integrated wound dressing device for treatment of an insertion site of percutaneous and drug delivery devices is described above. In each of the above embodiments, the different positions and structures of the present invention are described separately in each of the embodiments. However, it is the full intention of the inventor of the present invention that the separate aspects of each embodiment described herein may be combined with the other embodiments described herein. Those skilled in the art will appreciate that adaptations and modifications of the just-described preferred embodiment can be configured without departing from the scope and spirit of the invention. Therefore, it is to be understood that, within the scope of the appended claims, the invention may be practiced other than as specifically described herein.

Various modifications and alterations of the invention will become apparent to those skilled in the art without departing from the spirit and scope of the invention, which is defined by the accompanying claims. It should be noted that steps recited in any method claims below do not necessarily need to be performed in the order that they are recited. Those of ordinary skill in the art will recognize variations in performing the steps from the order in which they are recited. In addition, the lack of mention or discussion of a feature, step, or component provides the basis for claims where the absent feature or component is excluded by way of a proviso or similar claim language.

While various embodiments of the present invention have been described above, it should be understood that they have been presented by way of example only, and not of limitation. Likewise, the various diagrams may depict an example architectural or other configuration for the invention, which is done to aid in understanding the features and functionality that may be included in the invention. The invention is not restricted to the illustrated example architectures or configurations, but the desired features may be implemented using a variety of alternative architectures and configurations. Indeed, it will be apparent to one of skill in the art how alternative functional, logical or physical partitioning and configurations may be implemented to implement the desired features of the present invention. Also, a multitude of different constituent module names other than those depicted herein may be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the invention is described above in terms of various exemplary embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the invention, whether or not such embodiments are described and whether or not such features are presented as being a part of a described embodiment. Thus the breadth and scope of the present invention should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

A group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the invention may be described or claimed in the singular, the plural is contemplated to be within the scope thereof unless limitation to the singular is explicitly stated.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, may be combined in a single package or separately maintained and may further be distributed across multiple locations.

As will become apparent to one of ordinary skill in the art after reading this document, the illustrated embodiments and their various alternatives may be implemented without confinement to the illustrated examples. For example, block diagrams and their accompanying description should not be construed as mandating a particular architecture or configuration.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. An integrated wound dressing device comprising:
a gas-permeable transparent film layer having a bottom side, a top side and a perimeter;
the bottom side is coated with an adhesive impregnated with an antimicrobial agent;
the transparent film having a radius cutout between a central opening and the perimeter; and,
a liquid crystal temperature sensitive film with an adhesive placed on the top side of the transparent film layer near the central opening.

2. The dressing device of claim 1 further comprising a layer of double folded release paper below the bottom side of the transparent film layer.

3. The dressing device of claim 1 where there is a radial gap between the central opening and the liquid crystal sensitive film.

4. The dressing device of claim 1 where the liquid crystal temperature sensitive film is positioned around the central opening.

5. The dressing device of claim 1 where the liquid crystal temperature sensitive film is position around a half of the central opening.

6. An integrated wound dressing device comprising:
a gas-permeable transparent film layer having a bottom side, a top side and a perimeter;
the bottom side is coated with an adhesive layer impregnated with an antimicrobial agent and a non-toxic mixture of liquid crystal temperature sensitive esters; and,
the transparent film having a radius cutout between a central opening and the perimeter.

7. The dressing device of claim 6 further comprising a layer of double folded release paper below the bottom side of the transparent film layer.

8. The dressing device of claim 6 where the liquid crystal temperature sensitive esters are mixed with black carbonaceous powder.

9. The dressing device of claim 6 where the liquid crystal temperature sensitive esters are only present on a half of the transparent film adhesive layer.

10. An integrated wound dressing device comprising:
a foam layer having a perimeter;
said foam layer is permeated with a mixture of an antimicrobial agent and a non-toxic temperature sensitive liquid crystal esters; and,
the foam layer has a radius cutout between a central opening and the perimeter.

11. An integrated wound dressing device comprising:
a gas-permeable transparent film/cast containing dispersed aggregates of temperature sensitive liquid crystal esters having a bottom side and a top side;
the top side is smooth;
the bottom side is coated with an adhesive impregnated with an antimicrobial agent;
the transparent film/cast with aggregates of temperature sensitive liquid crystal esters having a radius cutout between a central opening and a perimeter.

12. The integrated wound dressing device of claim 11 further comprising a layer of double folded release paper below the bottom side of the transparent film/cast.

13. The integrated wound dressing device of claim 11 where the liquid crystal temperature sensitive crystal esters are mixed with black carbonaceous powder.

14. The integrated wound dressing device of claim 11 where liquid crystal temperature sensitive esters are only present in a half of the transparent film/cast.

15. The integrated wound dressing device of claim 1 where the liquid crystal temperate sensitive film or cast is applied in longitudinal stripes on the top side of the transparent film layer.

16. The integrated wound dressing device of claim 1 where the liquid crystal temperature sensitive film or cast is applied in a spotted-pattern on the top side of the transparent film layer.
